(19) United States
(12) Patent Application Publication
(10) Pub. No.: US 2006/0134171 A1
(43) Pub. Date: Jun. 22, 2006

(54) Agents which regulate, inhibit, or modulate the activity and/or expression of formyl peptide receptors as a unique means to both lower intraocular pressure and treat glaucomatous retinopathies/optic neuropathies

(75) Inventors: Loretta G. McNatt, Hurst, TX (US); Wan-Heng Wang, Grapevine, TX (US); Abbot F. Clark, Arlington, TX (US)

Correspondence Address: ALCON RESEARCH, LTD., R&D COUNSEL, Q-148, 6201 SOUTH FREEWAY, FORT WORTH, TX, 76134-2099, US

(73) Assignee: Alcon, Inc.

(21) Appl. No.: 11/292,158

(22) Filed: Dec. 1, 2005

Related U.S. Application Data
(60) Provisional application No. 60/636,511, filed on Dec. 16, 2004.

(51) Int. Cl. A61F 2/00

(52) U.S. Cl. 424/427

(57) ABSTRACT

The present invention provides a method for lowering intraocular pressure and providing neuroprotection to a patient in need thereof by administering a therapeutically effective amount of at least one non-nucleotide or non-protein agent that inhibits expression and/or signaling of FPR.

This application claims priority from the provisional application, U.S. Patent Application Ser. No. 60/636,511 filed Dec. 16, 2004.

### BACKGROUND OF THE INVENTION

1. Field of the Invention

The present invention relates to the field of ocular conditions involving neurodegeneration and/or elevated intraocular pressure. More specifically, the invention provides compositions that lower intraocular pressure and provide ocular neuroprotection.

2. Description of the Related Art

There are a number of ocular conditions that are caused by, or aggravated by, damage to the optic nerve head, degeneration of ocular tissues, and/or elevated intraocular pressure. For example, “glaucomas” are a group of debilitating eye diseases that are a leading cause of irreversible blindness in the United States and other developed nations. Primary Open Angle Glaucoma (“POAG”) is the most common form of glaucoma. The disease is characterized by the degeneration of the trabecular meshwork, leading to obstruction of the normal ability of aqueous humor to leave the eye without closure of the space (e.g., the “angle”) between the iris and cornea (Vaughan, D. et al., (1992)). A characteristic of such obstruction in this disease is an increased intraocular pressure (“IOP”), resulting in progressive visual loss and blindness if not treated appropriately and in a timely fashion. The disease is estimated to affect between 0.4% and 3.3% of all adults over 40 years old (Leske, M. C. et al. (1986); Bengtsson, B. (1989); Strong, N. P. (1992)). Moreover, the prevalence of the disease rises with age to over 6% of those 75 years or older (Strong, N. P., (1992)).

Glaucoma affects three separate tissues in the eye. The elevated IOP associated with POAG is due to morphological and biochemical changes in the trabecular meshwork (TM), a tissue located at the angle between the cornea and iris. Most of the nutritive aqueous humor exits the anterior segment of the eye through the TM. The progressive loss of TM cells and the build-up of extracellular debris in the TM of glaucomatous eyes lead to increased resistance to aqueous outflow, thereby raising IOP. Elevated IOP, as well as other factors such as ischemia, cause degenerative changes in the optic nerve head (ONH) leading to progressive “cupping” of the ONH and loss of retinal ganglion cells and axons. The detailed molecular mechanisms responsible for glaucomatous damage to the TM, ONH, and the retinal ganglion cells are unknown.

Twenty years ago, the interplay of ocular hypertension, ischemia and mechanical distortion of the optic nerve head were heavily debated as the major factors causing progression of visual field loss in glaucoma. Since then, other factors including excitotoxicity, nitric oxide, absence of vital neurotrophic factors, abnormal glial/neuronal interplay and genomics have been implicated in the degenerative disease process. The consideration of genomics deserves some discussion insofar as it may ultimately define the mechanism of cell death, and provide for discrimination of the various forms of glaucoma. Within the past 10 years, over 15 different glaucoma genes have been mapped and 7 glaucoma genes identified. This includes six mapped genes (GLC1A-GLC1F) and two identified genes (MYOC and OPTN) for primary open angle glaucoma, two mapped genes (GLC3A-GLC3B) and one identified gene for congentical glaucoma (CYP1B1), two mapped genes for pigmentary dispersion/pigmentary glaucoma, and a number of genes for developmental or syndromic forms of glaucoma (FOXC1, PITX2, LMX1B, PAX6).

Thus, each form of glaucoma may have a unique pathology and accordingly a different therapeutic approach to the management of the disease may be required. For example, a drug that effects the expression of enzymes that degrade the extracellular matrix of the optic nerve head would not likely prevent RGC death caused by excitotoxicity or neurotrophic factor deficit. In glaucoma, RGC death occurs by a process called apoptosis (programmed cell death). It has been speculated that different types of insults that can cause death may do so by converging on a few common pathways. Targeting downstream at a common pathway is a strategy that may broaden the utility of a drug and increase the probability that it may have utility in the management of different forms of the disease. However, drugs that effect multiple metabolic pathways are more likely to produce undesirable side-effects. With the advent of gene-based diagnostic kits to identify specific forms of glaucoma, selective neuroprotective agents can be tested with the aim of reducing the degree of variation about the measured response.

Current glaucoma therapy is directed to lowering IOP, a major risk factor for the development and progression of glaucoma. These therapies lower IOP, but they do not directly address the pathogenic mechanisms, and the disease continues to progress. Thus, what is needed is a therapeutic method for lowering IOP and/or providing neuroprotection to the optic nerve head and/or to retinal ganglion cells via pathogenic pathways.

### SUMMARY OF THE INVENTION

The present invention overcomes these and other drawbacks of the prior art by providing a method for lowering intraocular pressure and providing neuroprotection to a patient in need thereof by administering a therapeutically effective amount of a composition including at least one non-nucleotide or non-protein agent that inhibits expression or alters the function of formyl-peptide receptor (FPR), and a pharmaceutically acceptable carrier. In another aspect, the invention provides a method for lowering intraocular pressure by administering to a patient a therapeutically effective amount of an agent that inhibits expression or alters the function of FRP. Preferably, the compositions for use in the method of the invention will lower intraocular pressure that is elevated due to an increased expression of FPR or of a product of FPR signaling.

In preferred embodiments, the composition of the invention may be administered by topical application, intracamerally or via an implant. Typically, the total concentration of the FPR inhibitor in the composition of the invention will be from 0.01% to 2%. Generally, the treatment method of the invention will be most useful for a patient suffering from glaucoma, for example normal-tension glaucoma, or ocular hypertension.

The invention further provides a method for preventing the visual field loss associated with POAG by administering to a patient in need thereof a composition including a non-nucleotide or non-protein agent that modulates the expression and/or function of FPR such that intraocular pressure is controlled and protection is provided to retinal ganglion cells or to the optic nerve head.

In another embodiment, the present invention provides a composition for lowering intraocular pressure and providing neuroprotection in a patient in need thereof. Generally, the composition of the invention includes at least one agent that inhibits the expression and/or signaling of FPR and a pharmaceutically acceptable carrier. The total concentration of an inhibitor of FPR in the composition of the invention will preferably be from 0.01% to 2%.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. FPR-1 gene expression is elevated in glaucomatous vs. normal TM tissues. QPCR of FPR1 expression in 12 normal and 12 glaucoma TM tissues. Relative FPR1 mRNA level was normalized to 18 s. Each bar represents the mean+/−s.e.m for TM from 12 tissue donors. FPR mRNA in glaucoma tissues is significantly greater (2-fold) than that in normal tissues (p=0.005).

### DETAILED DESCRIPTION PREFERRED EMBODIMENTS

Glaucoma is a heterogeneous group of optic neuropathies that share certain clinical features. The loss of vision in glaucoma is due to the selective death of retinal ganglion cells in the neural retina that is clinically diagnosed by characteristic changes in the visual field, nerve fiber layer defects, and a progressive cupping of the ONH. One of the main risk factors for the development of glaucoma is the presence of ocular hypertension (elevated intraocular pressure, IOP). IOP also appears to be involved in the pathogenesis of normal tension glaucoma where patients have what is often considered to be normal IOP. The elevated IOP associated with glaucoma is due to elevated aqueous humor outflow resistance in the trabecular meshwork (TM), a small specialized tissue located in the iris-corneal angle of the ocular anterior chamber. Glaucomatous changes to the TM include a loss in TM cells and the deposition and accumulation of extracellular debris including plaque-like material. In addition, there also are changes that occur in the glaucomatous optic nerve head. In glaucomatous eyes, there are morphological and mobility changes in ONH glial cells. In response to elevated IOP and/or transient ischemic insults, there is a change in the composition of the ONH extracellular matrix and alterations in the glial cell and retinal ganglion cell axon morphologies.

Glaucomatous changes to the TM differ from fibrosis, which is associated with a wound healing response and generally involves inflammation and the subsequent proliferation of myofibroblasts. Tissue injury is recognized by the inflammatory system, which initiates a wound repair process by stimulating fibroblasts and angiogenesis. Dead or dying tissues/cells are replaced by scar tissue consisting initially of fibrin, which is subsequently replaced by excessive amounts of extracellular matrix material, particularly collagen.

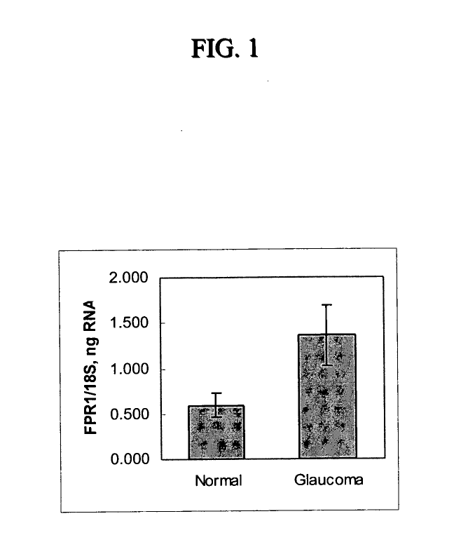

Recently we have identified that FPR expression at mRNA level was increased in a glaucoma RNA pool of trabecular meshwork tissues (12 donors) compared with that in normal TM tissues (9 donors) using Affymetric GeneChip technology. We further QPCR-quantitated the FPR expression using individual RNA from 12 glaucoma and 12 normal TM tissues. Again, FPR expression in the 12 glaucoma TM was significantly increased (2 fold, p=0.005) compared to that in the 12 normal TM (FIG. 1.) Formyl-peptide receptors in TM have not previously been described in the scientific literature. This is the first identification of this class of receptors in TM tissue, and the first demonstration of a significant upregulation of gene expression for these receptors in human ocular tissue.

FPRs belong to the seven transmembrane domain Gi-protein-coupled receptor (GPCR) gene family and have regulatory roles in Ca++ mobilization, anti-microbial and inflammatory responses, and amyloidogenic diseases and are expressed in many cell types. In humans there are three genes encoding two functional N-formylpeptide receptors, FPR (SEQ ID NO:1) and FPRL1 (FPR-like1, 70% identical to FPR in NT; SEQ ID NO:2), and a putative receptor FPRL2, (FPR-like 2, 70% identical to FPR or 82% identical to FPRL1 in NT; SEQ ID NO:3). All three genes cluster on chromosome 19q13.3. In contrast to three in humans, at least six members of FPR family (Fpr1, Fpr-rs1, Fpr-rs2, Fpr-rs3, Fpr-rs4, Fpr-rs5) have been identified in murine. Homology of the murine FPRs is more than 70% in NT compared to human FPR. In addition, many FPR agonists and antagonists have been identified and these discoveries suggest potential therapeutic use of agents that block or enhance FPR signaling in pathological conditions.

FPR is the high affinity receptor and binds the exogenous formyl peptide ligand, fMLF (formyl-methionyl-leucyl-phenylalanine) with Kd values in the picomolar to low nanomolar range and is activated by FMLF to mediate chemotactic and Ca²⁺ mobilizing responses in human phagocytic leukocytes. FPRL1 is a low affinity variant based on its activation only by high concentrations of FMLF. FPRL2 has only a limited expression profile and its function remains unclear. Formyl-peptide receptors are expressed in many cell types including phagocytic leukocytes, hepatocytes, astrocytes, microglial cells, immature dendritic cells, smooth muscle cells, endocrine cells and the tunica media of coronary cells. FPR localization has been demonstrated in a variety of human tissues and organs, including thyroid, adrenals, liver, and the nervous system. Becker et al. (1998) describes the presence of FPR in certain human organs, tissues, and cells, including the pigmented retinal epithelial cells, rods and cones, outer plexiform layer, and inner nuclear layer of the retinal, the iris epithelial layer and conjunctival epithelium, Bowman's and Descemet's membranes and the peripheral nerve Schwann cells. Nevertheless, the present inventors report, for the first time, the increased expression of FPR in glaucomatous TM.

Activation of formyl-peptide receptors results in increased cell migration, phagocytosis, release of proinflammatory mediators, and the signaling cascade culminates in heterologous desensitization of other seven transmembrane receptors (STM) including chemokine receptors CCR5 and CXCR4, two co-receptors for HIV-I (Shen, Proost et al. 2000). Thus, activation of FPR and FPRL1 by agonists subsequently interferes with cellular responses to a number of chemoattractants that use other unrelated STM receptors via heterologous receptor desensitization. Classically, FPR responds to chemotactic formylpeptides represented by (fMLF) produced by gram negative bacteria. The discovery of novel exogenous and host-derived FPR ligands in recent years suggests that FPR may also participate in biological processes other than anti-bacterial host responses and tissue injury. (Le, Murphy et al. 2002)

A wide variety of novel agonists that activate either or both FPR and FPRL1 have been identified. (Le, Murphy et al. 2002; Le, Yang et al. 2002) Formyl peptides released from damaged mitochondria activate FPR, and various non-formylated peptides, specifically HIV-1 envelope protein-derived peptides, annexin I and annexin I-derived peptides are FPR agonists. Small synthetic peptides selected from random peptide libraries, and host-derived peptide or lipid chemotractants activate FPR. Most of the chemoattractants specifically interact with the low affinity fMLF receptor FPRL1, and among a number of FPRL1 specific chemotactic agonists identified so far, at least three of them, the serum amyloid A (SAA), the 42 amino acid form of amyloid β (Aβ₄₂) and a peptide fragment of the human prion protein (PrP106-126), are amyloidogenic polypeptides. FPRL1 may play a significant role in proinflammatory responses seen in systemic amyloidosis, Alzheimer's disease (AD), and prion diseases, in which infiltration of activated mononuclear phagocytes at the sites of lesions is a common feature. (Su, Gong et al. 1999; Le, Oppenheim et al. 2001); (Becker, Forouhar et al. 1998) FPRL1 can function as both as a receptor for fMLF peptide and for the lipid mediator lipoxinA4 (LXA4) (Murphy, Ozcelik et al. 1992; Le, Oppenheim et al. 2001) FPR, but not FPRL1 has been shown to be a chemotatic receptor (Laudanna, C. et al, 1996, Science, 271, 981). Conversely, FPRL1, but not FPR, has been shown to be a functional lipoxin A4 receptor. (Fiore, S. et al, 1994, J exp med, 180, 253.; (Su, Gong et al. 1999)

Peptide derivatives as agonists and antagonists of FPR have been extensively reviewed and include both formylated and non-formylated peptides. (Le, Oppenheim et al. 2001; Le, Murphy et al. 2002; Le, Yang et al. 2002; Dalpiaz and Scatturin 2003; Dalpiaz, Spisani et al. 2003) Annexin I (lipocortin) peptides are endogenous FPR ligands that are FPR antagonists. None of the cited publications discusses the increased expression of FPR in glaucomatous TM or the use of inhibitors of FPR in the treatment of glaucoma.

Thus, in one aspect, the present invention provides a method for lowering IOP and providing neuroprotection to retinal ganglion cells by administering a composition including a non-nucleotide or non-peptidyl FPR inhibitor. It is further contemplated that the composition could include a compound that inhibits an agent which upregulates FPR. The therapeutic agent for the treatment of glaucoma will preferably be a small drug-like molecule, which affects one or more aspects of the FPR pathway. Preferred therapeutic agents are those that are: (1) inhibitors of FPR; (2) inhibitors of agents acting downstream of FPR action (i.e., inhibitors of FPR signaling) and/or (3) inhibitors of agents that is upregulate FPR gene or protein expression.

The agents of this invention, can be incorporated into various types of ophthalmic formulations for delivery to the eye (e.g., topically, intracamerally, or via an implant). The agents are preferably incorporated into topical ophthalmic formulations for delivery to the eye. The agents may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving an agent in a physiologically acceptable isotonic aqueous buffer. Further, the ophthalmic solution may include an ophthalmologically acceptable surfactant to assist in dissolving the agent. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the agent in a hydrophilic base prepared from the combination of, for example, carbopol-974, or the like, according to the published formulations for analogous ophthalmic preparations; preservatives and tonicity agents can be incorporated.

The agents are preferably formulated as topical ophthalmic suspensions or solutions, with a pH of about 4 to 8. The establishment of a specific dosage regimen for each individual is left to the discretion of the clinicians. The agents will normally be contained in is these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The agents can also be used in combination with other agents for treating glaucoma, such as, but not limited to, β-blockers, prostaglandin analogs, carbonic anhydrase inhibitors, α₂ agonists, miotics, and neuroprotectants.

The agent may be delivered directly to the eye (for example: topical ocular drops or ointments; slow release devices in the cul-de-sac or implanted adjacent to the sclera or within the eye; periocular, conjunctival, sub-Tenons, intracameral or intravitreal injections) or parenterally (for example: orally; intravenous, subcutaneous or intramuscular injections; dermal delivery; etc.) using techniques well known by those skilled in the art. The following are examples of possible formulations embodied by this invention.

| (a) Topical ocular formulation | wt. % |
|---|---|
| FPR Inhibitor | 0.005-5.0 |
| Tyloxapol | 0.01-0.05 |
| HPMC | 0.5 |
| Benalkonium chloride | 0.01 |
| Sodium chloride | 0.8 |
| Edetate disodium | 0.01 |
| NaOH/HCl | q.s. pH 7.4 |
| Purified water | q.s. 100 mL |

It is further contemplated that the compounds of the invention could be formulated in intraocular insert devices.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Example 1

### Increased Expression of FPR in Glaucomatous TM Cells and Tissues

Pooled RNA from trabecular meshwork tissue from 12 normal donors and 9 glaucoma donors was used to determine gene expression using the Affymetrix GeneChips set (HG-U133). FPR expression was increased in glaucoma TM tissue to 1.6 times that in normal TM tissue. To confirm this result, QPCR was conducted using individual RNA from 12 glaucoma and 12 normal TM tissues. The average FRP expression in glaucoma TM tissues was significantly greater (2-fold) than in normal tissue (p=0.005). (FIG. 1)

### EXAMPLE 2

### Induction of FPR in Cultured Cell Lines for Screening Compounds that Alter the Expression of FPR mRNA or Protein

HEK293 cells and U87 human glioma cells can be stably transfected with plasmids encoding FPR. (Ernst, Lange et al. 2004; Le, Iribarren et al. 2004) FPR mRNA expression can be determined by RT-QPCR. Cell surface expression of FPR protein can be detected by Flow cytometry using a monoclonal antibody to FRP. (Le, Iribarren et al. 2004)

### EXAMPLE 3

### Functional Analysis of FPR in Cultured Cells

Activation of FPR by its agonists initiates a cascade of signaling events that culminates in increased cell migration, phagocytosis, release of reactive oxygen intermediates, and new gene transcription. Chemotaxis assays, calcium mobilization assays, and receptor binding assays for formyl peptide receptors that have been well described can be used to characterize FPR response to agonists or antagonists. (Le, Hu et al. 2000; Ernst, Lange et al. 2004; Le, Iribarren et al. 2004). Human lung A549 cells expressing FPR respond to FMLF peptide agonist with elevation of expression of the acute phase protein, fibrinogen. (Rescher, Danielczyk et al. 2002) HepG2 hepatoma cells also express FPR and can be used for evaluating both agonistic and antagonistic ligands for the receptor. (Rescher, Danielczyk et al. 2002) The promyelocytic human leukemia HL-60 cell line when chemically differentiated express active FPR. This cell line was used for a large SAR screening of cyclosporins for FPR inhibition. (Loor, Tiberghien et al. 2002) HL-60 cells express the low affinity FPRL1 and can be used for to evaluate differential activation or inhibition of the formylpeptide receptors. (Hoyle and Freer 1984; Bae, Song et al. 2003; Bae, Yi et al. 2003; Dalpiaz and Scatturin 2003)

FPR function can be assayed by a ligand-induced granule enzyme release using HL-60 cells as described by Loor et al. (Loor, Tiberghien et al. 2002) The release of N-acetyl-β-D-glucosamimidase was measured upon stimulation of HL-60 cells with FMLF in the presence of a range of concentrations of potential antagonists. The assay uses the enzyme substrate p-nitrophenyl-N-acetyl-β-D-glucosaminide, and glucosamimidase activity is measured by the release of p-nitrophenol which is measured spectrophotometrically.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents

Books

Other Publications
Bae, Y. S., J. Y. Song, et al. (2003). “Differential activation of formyl peptide receptor signaling by peptide ligands.” Mol Pharmacol 64(4): 841-7.
Bae, Y. S., H. J. Yi, et al. (2003). “Differential activation of formyl peptide receptor-like 1 by peptide ligands.” J Immunol 171(12): 6807-13.
Becker, E. L., F. A. Forouhar, et al. (1998). “Broad immunocytochemical localization of the formylpeptide receptor in human organs, tissues, and cells.” Cell Tissue Res 292(1): 129-35.
Bengtsson, B., Br. J. Ophthalm. 73:483-487 (1989).
Dalpiaz, A. and A. Scatturin (2003). “Peptide derivatives as agonists or antagonists of formylpeptide receptors: analysis of their effects on neutrophils.” Mini Rev Med Chem 3(3): 167-73.
Dalpiaz, A., S. Spisani, et al. (2003). “Studies on human neutrophil biological functions by means of formyl-peptide receptor agonists and antagonists.” Curr Drug Targets Immune Endocr Metabol Disord 3(1): 33-42.
Ernst, S., C. Lange, et al. (2004). “An annexin 1 N-terminal peptide activates leukocytes by triggering different members of the formyl peptide receptor family.” J Immunol 172(12): 7669-76.
Fiore, S. et al., J. Exp. Med. 180:253 (1994).
Greve, M. et al., Can. J. Ophthalm. 28:201-206 (1993).
Hoyle, P. C. and R. J. Freer (1984). “Isolation and reconstitution of the N-formylpeptide receptor from HL-60 derived neutrophils.” FEBS Lett 167(2): 277-80.
Laudanna, C. et al., Science 271:981 (1996).
Le, Y., J. Hu, et al. (2000). “Expression of functional formyl peptide receptors by human astrocytoma cell lines.” J Neuroimmunol 111(1-2): 102-8.
Le, Y., P. Iribarren, et al. (2004). “Silencing the formylpeptide receptor FPR by short-interfering RNA.” Mol Pharmacol 66(4): 1022-8.
Le, Y., P. M. Murphy, et al. (2002). “Formyl-peptide receptors revisited.” Trends Immunol 23(11): 541-8.
Le, Y., J. J. Oppenheim, et al. (2001). “Pleiotropic roles of formyl peptide receptors.” Cytokine Growth Factor Rev 12(1): 91-105.
Le, Y., Y. Yang, et al. (2002). “Receptors for chemotactic formyl peptides as pharmacological targets.” Int Immunopharmacol 2(1): 1-13.
Leske, M. C. et al., Amer. J. Epidemiol. 113:1843-1846 (1986).
Loor, F., F. Tiberghien, et al. (2002). “Cyclosporins: structure-activity relationships for the inhibition of the human FPR1 formylpeptide receptor.” J Med Chem 45(21): 4613-28.
Murphy, P. M., T. Ozcelik, et al. (1992). “A structural homologue of the N-formyl peptide receptor. Characterization and chromosome mapping of a peptide chemoattractant receptor family.” J Biol Chem 267(11): 7637-43.
Rescher, U., A. Danielczyk, et al. (2002). “Functional activation of the formyl peptide receptor by a new endogenous ligand in human lung A549 cells.” J Immunol 169(3): 1500-4.
Shen, W., P. Proost, et al. (2000). “Activation of the chemotactic peptide receptor FPRL1 in monocytes phosphorylates the chemokine receptor CCR5 and attenuates cell responses to selected chemokines.” Biochem Biophys Res Commun 272(1): 276-83.
Shepard et al., IOVS 42:3173 (2001).
Strong, N. P. Ophthalm. Physiol. Opt. 12:3-7 (1992).
Su, S. B., W. Gong, et al. (1999). “A seven-transmembrane, G protein-coupled receptor, FPRL1, mediates the chemotactic activity of serum amyloid A for human phagocytic cells.” J Exp Med 189(2): 395-402.
Vaughan. D. et al., (1992).
Wang et al., Mol. Vis. 7:89-94 (2001).

We claim:

1. A method for lowering intraocular pressure and providing neuroprotection to a patient in need thereof, said method comprising administering a therapeutically effective amount of a composition comprising at least one non-nucleotide or non-protein agent that inhibits expression, signaling or biological functions of formylpeptide receptor (FPR), and a pharmaceutically acceptable carrier.

2. The method of claim 1, wherein said administering is by topical application, intracamerally or via an implant.

3. The method of claim 1, wherein the total concentration of said FPR inhibitor in said composition is from 0.01% to 2%.

4. The method of claim 1, wherein said patient suffers from glaucoma or ocular hypertension.

5. The method of claim 4, wherein said glaucoma is normal-tension glaucoma.

6. A method for lowering intraocular pressure in a patient in need thereof, said method comprising administering a therapeutically effective amount of a composition comprising at least one non-nucleotide or non-protein agent that inhibits expression, signaling, or biological functions of connective tissue growth factor (FPR), and a pharmaceutically acceptable carrier.

7. The method of claim 6, wherein said administering is by topical application, intracamerally or via an implant.

8. The method of claim 6, wherein the total concentration of said FPR inhibitor in said composition is from 0.01% to 2%.

9. The method of claim 6, wherein said patient suffers from glaucoma or ocular hypertension.

10. The method of claim 9, wherein said glaucoma is normal-tension glaucoma.

11. A method for preventing the visual field loss associated with Primary Open Angle Glaucoma (POAG), said method comprising administering to a patient in need thereof a composition comprising a non-nucleotide or non-protein agent that modulates the expression of FPR such that intraocular pressure is controlled and protection is provided to retinal ganglion cells or to the optic nerve head.

12. A composition for lowering intraocular pressure and providing neuroprotection in a patient in need thereof, said composition comprising at least one agent that inhibits the expression, signaling, or biological functions of FPR and a pharmaceutically acceptable carrier.

13. The composition of claim 12, wherein the total concentration of said FPR inhibitor in said composition is from 0.01% to 2%.
